(19) United States
(12) Patent Application Publication
(10) Pub. No.: US 2015/0018741 A1
(43) Pub. Date: Jan. 15, 2015

(54) METHOD AND APPARATUS FOR FOOT AMENDMENT

(71) Applicant: Aharon LIBERSON, (US)

(72) Inventors: Shmuel Lieberson, Haifa (IL); Anton Uskov, Haifa (IL); Eugeny Liberson, Ramat Gan (IL)

(21) Appl. No.: 14/357,576

(22) PCT Filed: Nov. 12, 2012
(86) PCT No.: PCT/IL12/50454

§ 371 Date: May 12, 2014

Related U.S. Application Data
(60) Provisional application No. 61/559,082, filed on Nov. 13, 2011.

(51) Int. Cl. A61F 5/01

(52) U.S. Cl. CPC A61F 5/0127
USPC 602/28

(57) ABSTRACT

A method for amending a foot of a person, comprising disposing one or more elevations under the foot, wherein an elevation is disposed under an at least one metatarsal bone of the foot and either alternatively or additionally an elevation is disposed lengthwise diagonally under the heel of the foot, and applying a downwards force on the foot, thereby warping the foot over the elevations and amending the foot arch, and an apparatus and kit for performing the same.

### BACKGROUND

The present disclosure generally relates to treatment of foot disorders and more specifically to a modification of the foot arch.

Orthotics are generally built to function as padding to relieve the pressure under the heel, allow homogenous pressure spread on the foot surface, and plantar fascia tension relief as reported, for example, in Abnormal biomechanics. In Donatelli R A, Ed: Biomechanics of the Foot and Ankle. 2nd ed. Philadelphia: F A Davis; 1996:34-72. For instance, padding under the 1st metatarsal and lateral part of the heel in tailor-made insoles.

Some treatments involve shortening or elongation of windlass mechanism by the insoles as reported, for example, in Journal of Athletic Training 2004 January-March; 39(1): 77-82.

Some other methods or apparatus were devised, as briefly exemplified below.

U.S. Pat. No. 1,462,534 reports on devices for supporting the arch or instep of the human foot to correct what is familiarly known as a flat foot and to prevent a tendency in that direction.

U.S. Pat. No. 1,374,669 reports foot supporters to provide flexible braces or bands adapted to fit closely about the ankle and instep of the foot and to be adjusted to compensate for variations or changes.

U.S. Pat. No. 1,465,970 reports an arch-support which to be o effective in supporting the arch when a shoe is not being worn.

U.S. Pat. No. 1,746,865 reports means that may be worn in the shoe and act to restore the in-turned great-toe to its normal position.

US Application No. 2004/0033874 reports footwear comprising a support member.

US Application No. 2009/0247921 reports a foot or ankle wrap, and method of treating infirmities and abnormalities of the foot or ankle by wrapping the foot or ankle with one or more straps.

US Application No. 2011/0047831 reports an element for attachment to the bottom surface of a sole of a shoe.

US Application No. 2011/0107501 reports a sock or similar garment for preventing leg alignment problems.

### SUMMARY

One exemplary embodiment of the disclosed subject matter is method for amending a foot of a person, comprising disposing elevations under the foot, wherein an at least one first elevation is disposed under an at least one metatarsal bone of the foot and an at lest one second elevation is disposed lengthwise diagonally under the heel of the foot, and applying a downwards force on the foot, thereby warping the foot over the elevations and amending the foot arch.

Another exemplary embodiment of the disclosed subject matter is a apparatus for amending a foot of a person, comprising at least two elevations for disposing under the foot, and an at least one fastener for securing the elevations in place under the foot.

Yet another exemplary embodiment of the disclosed subject matter is a kit for amending a foot of a person, comprising at least two elevations for disposing under the foot, and an at least one fastener for securing the elevations in place under the foot.

Yet another exemplary embodiment of the disclosed subject matter is a method for amending a foot of a person, comprising disposing an at least one elevation under the foot, disposed under an at least one metatarsal bone of the foot or disposed under the heel of the foot, and applying a downwards force on the foot, thereby warping the foot over the elevations and amending the foot arch.

In the context of the present disclosure, without limiting, a ‘foot’ implies a human foot, and referring to a person implies the human having the foot. For brevity and clarity and without limiting, in the present disclosure, the person is assumed to be a male.

In the context of the present disclosure, without limiting, an ‘arch’ of the foot’ or ‘arch’ generally imply the combination and/or the shape of the combination of the medial longitudinal arch, the lateral longitudinal arch and the transverse arch, unless otherwise specified.

In the context of the present disclosure, without limiting, a ‘flat foot’ implies the customary meaning and/or lack of or a reduced curvature of the arch relative to a proper or a sufficient arch.

In the context of the present disclosure, without limiting, referring to a length of a foot implies a general direction from the toes to the heel, and likewise, referring to a width or a lateral direction implies a general direction generally perpendicular to the foot length.

In the context of the present disclosure, without limiting, referring to ‘lengthwise diagonally’ implies a direction obliquely or slanted relative to the length of a foot.

In the context of the present disclosure, without limiting, the region at or close to the toes relative to the rest of the foot is referred to as the front of the foot or forefoot, and the region at or close to the heel relative to the rest of the foot is referred to as the back or rear of the foot or hindfoot, and a behind relations implies towards the rear of the foot.

For brevity and clarity and without limiting, referring to a low part of or beneath or under a foot is with respect to a standing posture of the person, and accordingly referring to under or beneath a component disposed under the foot implies towards the ground or floor rather than between the component and the foot, and accordingly upwards and top refer to the opposite direction or orientation. Likewise, the term downwards is in a general direction to the ground with respect to a standing posture of the person.

As used herein, the terms ‘about’ or ‘close’ imply at or in a region of or close to a location or part of a foot relative to other parts or regions of the foot.

For brevity and clarity and without limiting, in the present disclosure a left foot is assumed to be referenced, unless otherwise specified.

### BRIEF DESCRIPTION OF THE DRAWINGS

Some non-limiting exemplary embodiments or features of the disclosed subject matter are illustrated in the following drawings.

Identical or duplicate or equivalent or similar structures, elements, or parts that appear in one or more drawings are generally labeled with the same reference numeral, optionally with an additional letter or letters to distinguish between similar objects or variants of objects, and may not be repeatedly labeled and/or described.

Dimensions of components and features shown in the figures are chosen for convenience or clarity of presentation and are not necessarily shown to scale or true perspective. For convenience or clarity, some elements or structures are not shown or shown only partially and/or with different perspective or from different point of views.

References to previously presented elements are implied without necessarily further citing the drawing or description in which they appear.

FIG. 1A schematically illustrates a medial view of a left foot with elevations disposed under the foot behind the small toes (not shown) and about the heel, according to exemplary embodiments of the disclosed subject matter;

FIG. 1B schematically illustrates a lateral view of a left foot with elevations disposed under the foot behind the small toes (not shown) and about the heel, according to exemplary embodiments of the disclosed subject matter;

FIG. 1C schematically illustrates plantar view of a left foot with elevations lengthwise diagonally disposed under the foot behind the small toes and about the heel, according to exemplary embodiments of the disclosed subject matter;

FIG. 1D schematically illustrates lateral view of a left foot with a sock having elevations lengthwise diagonally disposed under the foot behind the small toes and about the heel, according to exemplary embodiments of the disclosed subject matter;

FIG. 2 schematically illustrates a perspective side view of a box-figure representation of a foot, according to exemplary embodiments of the disclosed subject matter;

FIG. 3A schematically illustrates a perspective front view of a box-figure representation of FIG. 2 with elevations lengthwise diagonally disposed under the foot as a flat foot behind the small toes on the metatarsal bones and about the heel, respectively, according to exemplary embodiments of the disclosed subject matter;

FIG. 3B schematically illustrates a perspective front view of a box-figure representation of FIG. 3A of a flat foot after standing, according to exemplary embodiments of the disclosed subject matter;

FIG. 4A schematically illustrates a perspective front view of a box-figure representation of FIG. 2 with elevations lengthwise diagonally disposed under the foot behind the large toe or toes on the metatarsal bones and about the heel, respectively, according to exemplary embodiments of the disclosed subject matter;

FIG. 4B schematically illustrates a perspective front view of a box-figure representation of FIG. 4A of a foot, having an arch, after standing, according to exemplary embodiments of the disclosed subject matter;

FIG. 5 outlines operations for applying correction to a flat foot, according to exemplary embodiments of the disclosed subject matter;

FIG. 6 outlines operations for relieving disorder syndromes of a foot, according to exemplary embodiments of the disclosed subject matter;

FIG. 7 outlines operations for warping a foot, according to exemplary embodiments of the disclosed subject matter; and

FIG. 8 schematically illustrates a kit for amending a foot, according to exemplary embodiments of the disclosed subject matter.

### DETAILED DESCRIPTION

A general technical problem dealt by the disclosed subject matter is applying a corrective force or pressure to counteract a disorder or a deformation of a foot.

One technical problem dealt by the disclosed subject matter is applying a corrective force or pressure in order to increase a foot arch curvature or in order to remedy a flat foot disorder.

Another technical problem dealt by the disclosed subject matter is applying a corrective force or pressure in order to decrease a foot arch curvature thereby reducing or relieving conditions such as inflammation and thickening of plantar fascia and/or compression of nerves adjacent to fascia in Tarsal Tunnel Syndrome and/or enthesopathy Tendo Achilles.

A general solution according to the disclosed subject matter is a pair of elevations, such as pads, disposed lengthwise diagonally under the foot, one elevation at the front and one at back of the foot. Thus, when the person stands or walks his weight compels the arch to warp and vary due to the diagonal or oblique relative positions of the elevations under the foot, thereby amending the arch curvature to provide a healing effect and/or therapy and/or relief of pain or disorder or syndromes.

In some embodiments, the solution includes one or more fasteners in order to keep the elevations intact in place even when the person stands walks or runs. For example, elastic straps that wrap under the elevations and over and around the foot or a part thereof, adhesive stripes disposed under the elevation, or applying bonding agent that removably fasten the elevations to the foot or wearing on the foot a garment such as a sock or a part thereof, half-sock or half-toe sock, optionally comprising therein elastic straps and optionally comprising the elevations therein.

In some embodiments, the height or thickness and/or shape and/or location and/or fastening and/or triggering force or pressure of the elevations is varied during the therapy session, such as periodically or by time intervals, providing a gradual variation of the arch.

In some embodiments, the elevations and optionally further the fasteners are devised to enable wearing shoes and potentially allowing to proceed with daily routine.

One technical solution according to the disclosed subject matter is a pair of elevations, such as pads, disposed under the foot, a first elevation about or behind the 5^{th} and/or the 4^{th} and/or also the 3^{rd} metatarsal bones, that is, behind the small toes, and a second elevation is disposed about and under the heel lengthwise diagonally from the first elevation. That is, the first elevation is disposed beneath and about the left frontal region of the foot or midway between the front and back of the foot, and the second elevation is disposed beneath and about the right rear region of the foot under the heel or side thereof.

As the person walks or stands, his weight applies a force or a pressure compelling the arch to increase. For example, the weight applies a force or a pressure on a flat foot counteracting the flatness of the foot and, at least gradually, improving the arch shape towards a proper or a sufficient curvature.

In some embodiments, the solution includes one or more fasteners in order to keep the elevations intact in place even when the person stands walks or runs as described above.

Another technical solution according to the disclosed subject matter is a pair of elevations, such as pads, disposed under the foot, a first elevation about or behind the 1^{st} and/or the 2^{nd} and/or also the 3^{rd} metatarsal bones, that is, behind the large toe or toes, and a second elevation is disposed under the heel lengthwise diagonally from the first elevation. That is, the first elevation is disposed beneath and about the right frontal region of the foot or midway between the front and back of the foot, and the second elevation is disposed beneath and about the left rear region of the foot under the heel or side thereof.

As the person walks or stands, his weight applies a force or a pressure thereby shifting the foot laterally causing an artificial insufficiency of the first ray and compelling the arch to decrease. For example, the weight applies a force or a pressure on a having disorder syndrome or pain such as due to damaged tendons and/or inflammation and thickening of plantar fascia and/or compression of nerves adjacent to fascia in Tarsal Tunnel Syndrome and/or enthesopathy Tendo Achilles, relieving the pain and/or symptoms and, at least potentially and/or gradually counteracting the disorder and/or deformation of the foot.

In some embodiments, the solution includes one or more fasteners in order to keep the elevations intact in place even when the person stands walks or runs as described above.

It is noted that the solutions do are not necessarily limited to a pair of elevation and, in some embodiments, one or more solutions comprise one elevation or more than two elevations. In some embodiments, the elevations are connected therebetween, optionally with semi-rigid or a flexible member, providing for convenient and repetitive positioning of the pads. In some embodiments, a fastener such as a sock comprises the elevations so that by wearing the sock the elevations are disposed at the intended position or at least sufficiently close the intended position, wherein the sock is flexible akin to conventional socks.

One potential technical effect of the disclosed subject matter is bending or twist or warping the foot to repair, at least partially, an anatomical disorder such as a flat foot.

Another potential technical effect of the disclosed subject matter is bending or twist or warping the foot to relieve, at least partially, a pain and/or syndromes of the foot, while wearing shoes.

A general non-limiting overview of practicing the present disclosure is presented below. The overview outlines exemplary practice of embodiments of the present disclosure, providing a constructive basis for variant and/or alternative and/or divergent embodiments, some of which are subsequently described.

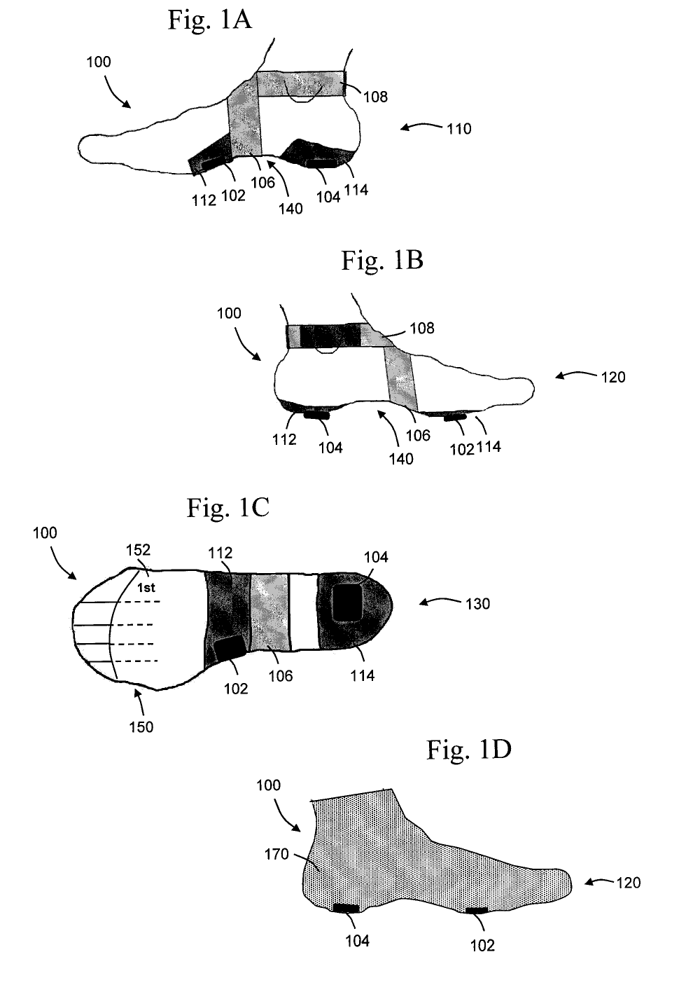

FIG. 1A schematically illustrates a medial view 110 of a foot 100 with elevation 102 and elevation 104 disposed lengthwise diagonally (not shown) under foot 100 behind the small toes (not shown) and about the heel, respectively, according to exemplary embodiments of the disclosed subject matter,

FIG. 1B schematically illustrates a lateral view 120 of foot 100 with elevation 102 and elevation 104 disposed lengthwise diagonally (not shown) under foot 100 behind the small toes (not shown) and about the heel, respectively, according to exemplary embodiments of the disclosed subject matter.

FIG. 1C schematically illustrates an plantar view 130 of foot 100, having metatarsal bones 150 where the 1^{st} metatarsal bone 152 is indicated, with elevation 102 and elevation 104 disposed lengthwise diagonally under foot 100 behind the small toes and about the heel or the side of the heel, respectively, according to exemplary embodiments of the disclosed subject matter.

FIG. 1D schematically illustrates a lateral view 120 of foot 100 wearing a sock 170 having elevation 102 and elevation 104 disposed lengthwise diagonally (not shown) under foot 100 behind the small toes (not shown) and about the heel, respectively, according to exemplary embodiments of the disclosed subject matter.

In some embodiments, the shape, width and height, extent under the foot, of the elevations, such as elevation 102 and elevation 104, are designed or made to achieve, at least partially, a remedy to foot 100 as described below.

In some embodiments, the elevations, such as elevation 102 and elevation 104, are made of adequately and/or sufficiently compressible material to adjust to the foot shape at locations thereof. For example, silicone pads, sponge, foamed plastic or any combination thereof or any other material such as air-filled compartment.

In some embodiments, the elevation comprises and/or is made of an inflatable compartment, optionally or additionally allowing inflating the compartment to a certain height and/or size, for example, as required by the foot condition.

In some embodiments, an elevation, such as elevation 102 and/or elevation 104, comprises or is made of or constitutes a plurality of elevations, thereby allowing and/or enabling a simultaneous and/or an alternative therapy, at least partially, of a plurality of disorders or conditions. For example, using four elevations allows simultaneous therapy, at least partially, of pronation, supination, pes cavus and pes planus (flat foot).

In some embodiments, the elevation comprises or is made of a plurality of inflatable compartments, optionally or additionally allowing inflating certain compartments to certain heights and/or sizes, thereby enabling elevation at a plurality of locations under the foot and providing simultaneous effects on a plurality of conditions. Namely, the plurality of compartments is effective similarly or equivalently to a plurality of elevations.

In some embodiments, when gradual therapy is required or used, then instead of replacing elevations the inflatable compartment or compartments are inflated, such as by inflating or deflating the inflatable compartment or compartments, providing continuous adjustments of the elevation or elevations.

In some embodiments, an inflatable compartment such as described above, or other structures such as pads, may be used as cushion and/or immobilization for a part of the foot, such as the rear of the foot, optionally for immobilizing a fracture in the foot.

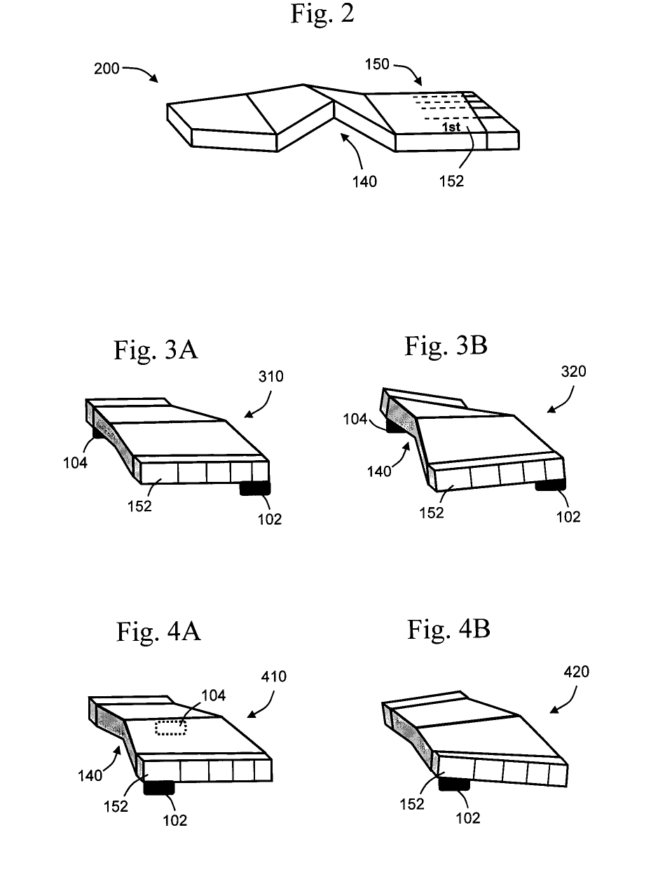

For brevity and clarity, as described herein, without limiting and unless otherwise specified, foot 100 is a left foot where foot 100 may also be represented by other constructs such as box-figure as in FIG. 2 and FIGS. 3A-4B discussed later on.

The locations of the elevations and/or size thereof and/or height thereof are designed or made such that when the person stands on the foot the arch curvature is increased, that is, deepened. For example, if foot 100 is a flat foot or having a too shallow curvature by standing on the elevations as shown, for example, in the views of FIGS. 1A-1C then the arch 140 is forced by the weight of the person to curve upwards and, optionally and/or potentially, also laterally.

FIG. 2 schematically illustrates a perspective side view of a box-figure representation 200 of foot 100 having metatarsal bones 150 where the 1^{st} metatarsal bone 152 is indicated, according to exemplary embodiments of the disclosed subject matter.

FIG. 3A schematically illustrates a perspective front view 310 of a box-figure representation 200, with elevation 102 and elevation 104 lengthwise diagonally disposed under foot 100 as a flat foot behind the small toes on the metatarsal bones and about the heel, respectively, according to exemplary embodiments of the disclosed subject matter.

FIG. 3B schematically illustrates a perspective front view 320 of a box-figure representation 200 of a flat foot after standing, according to exemplary embodiments of the disclosed subject matter, where the foot is warped and the curvature of arch 140 is increased or enhanced.

In some embodiments, in order to gradually enhance the curvature of the arch, the locations and/or size and/or height of the elevations, such as elevation 102 and elevation 104, are modified during a therapy session, enabling or allowing the curvature to gradually assume a proper and/or a sufficient curvature.

In some cases, a foot may have a disorder syndrome or pain such as due to stretched tendons and/or inflammation and thickening of plantar fascia and/or compression of nerves adjacent to fascia in Tarsal Tunnel Syndrome and/or enthesopathy Tendo Achilles and/or pes cavus, and/or Tarsal Tunnel and/or Plantar Fasciitis collectively also referred to, without limiting, as ‘disorder syndromes’.

In some embodiments, in order to relieve the pain due to the disorder syndromes the foot is warped, thereby reducing the curvature of the arch and relieving, at least partially, at least one of the disorder syndromes such as relaxing and/or immobilizing a tension of Tendo Achilles

FIG. 4A schematically illustrates a perspective front view 410 of a box-figure representation 200 with elevation 102 and elevation 104 lengthwise diagonally disposed under foot 100, having arch 140, behind the large toe or toes on the metatarsal bones and about the heel, respectively, according to exemplary embodiments of the disclosed subject matter.

FIG. 4B schematically illustrates a perspective front view 120 of a box-figure representation 200 of a foot, having an arch, after standing, according to exemplary embodiments of the disclosed subject matter, where the foot is warped and the curvature of arch 140 is decreased thereby amending the foot by reducing, at least potentially and/or partially, at least one of the disorder syndromes such as relaxing a tension of Tendo Achilles.

In some embodiments, the locations and/or size and/or height of the elevations, such as elevation 102 and elevation 104, are modified during a therapy session, enabling or allowing the foot anatomy to heal at least to some extent and assume a proper and/or a sufficient condition, and/or preventing pain to the person, at least to some extent.

In some embodiments, an elevation disposed under the foot front, such as elevation 102, and/or an elevation disposed at and under the foot rear, such as elevation 104, comprises more than one elevation such as two or more pads.

In some embodiments, an elevation disposed under the heel front, such as elevation 102, and an elevation disposed at and under the foot rear, such as elevation 104, are connected therebetween. For example, pads are attached to a structure that fits or locks on the foot thereby conveniently and/or repetitively placing the pads in proper locations, where such a structure is, in some embodiments, a sock or a half-sock.

In some embodiments, the sock or half-sock, such as sock 170 illustrated schematically in FIG. 1D, is made of a stretchable fabric and/or an elastic fabric and has elevations, such as elevation 102 and elevation 104, fastened to and/or built in the fabric for placing in the intended locations under the foot. Consequently, when the sock, or half-sock for that matter, is worn on the foot the sock is stretched over the foot thereby adhering and/or securing the elevations lengthwise diagonally under the foot, one elevation at the front and one at back of the foot.

In some embodiments, only a single elevation is disposed under the foot generally as or similar to as described for two or more elevations, and the foot or arch is warped due to the single elevation, generally depending on the position of the single elevation under the foot and/or the anatomy and/or disorder of the foot.

It is noted that the structures of FIG. 2 and FIGS. 3A-4B are intended to convey a practice of the disclosed subject matter without necessarily depicting credible forms of a human foot.

In some embodiments, in order to keep or secure the elevations, such as elevation 102 and elevation 104, in place and prevent dislocation of the elevations, one or more fasteners are used.

For example, referring to the views of FIGS. 1A-1C, elastic and/or adhesive bands, such as a band 112 and a band 114, are used to hold elevation 102 and elevation 104, respectively, where the bands are optionally removably adhesive.

Optionally or additionally, other fasteners may be used, for example, bonding agent that removably fasten the elevations to the foot, or wearing a garment such as a sock or a part thereof, or a half-sock or a half-toe sock where the socks optionally comprising therein elastic or adhesive straps.

In some embodiments, when the elevations are attached or a part of a structure that fits or locks on the foot, the structure may be fastened to the foot such as by an elastic strap or an adhesive band.

In some embodiments, the fastening of the elevations, potentially at least, allow the person to wear shoes and stand and/or walk and/or run, keeping the elevations in place and thus conducting the therapy during daily or routine activities.

In some embodiments, in order to further increase the posture and/or stability of the foot while conducting the therapy by the elevations, as described above, either with shoes or without shoes, the foot is wrapped with straps. For example, a strap 106 and/or a strap 108 as shown in FIGS. 1A-1C. In some embodiments, the straps are elastic thereby applying pressure on the foot where the straps are disposed thereon.

Optionally or additionally, other members may be used for improving the posture and/or stability of the foot while conducting the therapy, such as an elastic sock or ankle sock or a half-sock.

In some embodiments, applying elastic straps or other members as described above stimulate and/or activate muscles of the foot thereby strengthening the muscles and/or posture of the foot regardless whether elevations are disposed under the foot or not.

By having the person standing and/or walking and/or running then due to the person's weight the muscles of the foot are stimulated or trained, optionally aided by the fasteners and/or by an elastic member as a band under the arch and/or the straps.

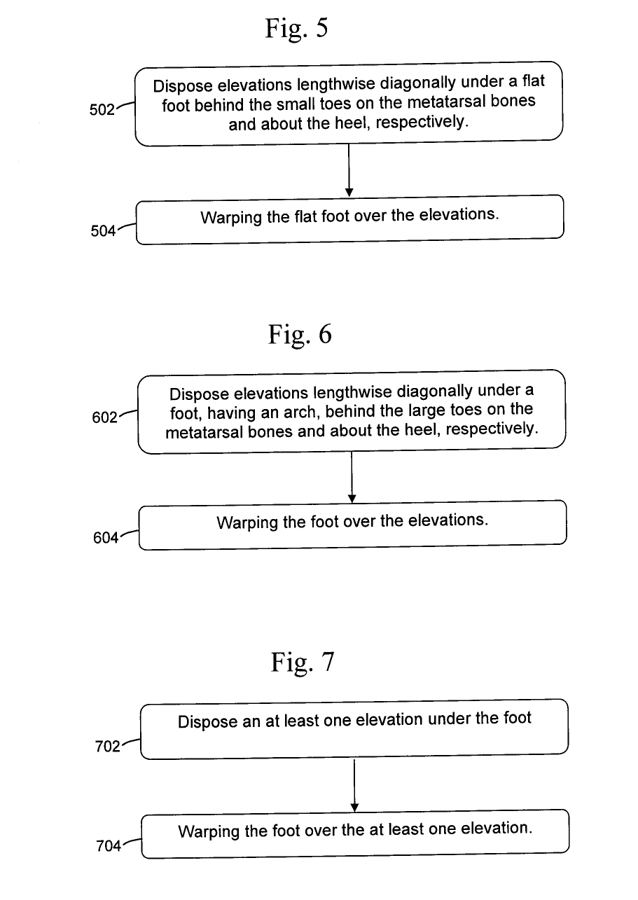

FIG. 5 outlines operations for applying correction to a flat foot, according to exemplary embodiments of the disclosed subject matter.

At 502 elevations are disposed or applied lengthwise diagonally under a flat foot behind the small toes or toe on the metatarsal bones and about the heel, respectively.

In some embodiments, the elevations are kept intact in place by fasteners that prevent, at least partly, movement of the elevations.

At 504 the flat foot is warped over the elevations. In some embodiments, the warping is due to force over the flat foot such as by the weight of the person. In some embodiments, warping of the flat foot over the elevations increases or enhances the curvature of the arch of the foot.

FIG. 6 outlines operations for relieving disorder syndromes of a foot, according to exemplary embodiments of the disclosed subject matter.

In some cases and/or embodiments, the disorder syndromes comprise one or more of stretched tendons and/or inflammation and thickening of plantar fascia and/or compression of nerves adjacent to fascia in Tarsal Tunnel Syndrome and/or enthesopathy Tendo Achilles and/or Flexor Hallucis Longus Tendonitis.

At 602 elevations are disposed or applied lengthwise diagonally under a foot, having an arch, behind the large toe or toes on the metatarsal bones and about the heel, respectively.

At 604 the foot is warped over the elevations. In some embodiments, the warping is due to force over the foot such as by the weight of the person. In some embodiments, warping of the foot over the elevations decreases the curvature of the arch of the foot, thereby relieving, at least partially, at least one of the disorder syndromes.

In some embodiments, only a single elevation instead of two or more are used, such as at the front of the foot or the rear of the foot.

FIG. 7 outlines operations for warping a foot by an at least one elevation, according to exemplary embodiments of the disclosed subject matter.

At 702 an at least one elevation is disposed under the foot, such as at and under the heel or at or behind a metatarsal bone or metatarsal bones.

At 704 under a pressure such as the weight of the person the foot is warped over the at least one elevation thereby increasing the curvature of the foot arch and/or decreasing the curvature of the foot arch.

In some embodiments, the methods of FIG. 5 and/or FIG. 6 and/or FIG. 7 further comprise applying or disposing one or more fasteners to prevent movement of the elevation or elevations.

In some embodiments, the methods of FIG. 5 and/or FIG. 6 and/or FIG. 7 are varied from time to time, such as by varying the shape and/or size and/or height of the elevations, thereby applying gradual modification and/or correction of the foot shape and/or disorder such that the foot, at least potentially, gradually acquire a sufficiently proper shape and/or relief of at least one of disorder syndromes or symptoms.

In some embodiments of the present disclosure, apparatus and/or items used for amending a foot arch and optionally for further stabilizing the foot are provided in a kit.

For example, an assortment of pads of various dimension to fit for different sizes of feet and/or amount of amendment and/or for gradual application of the amendment.

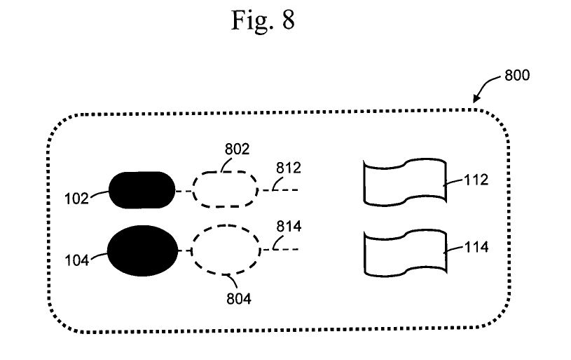

FIG. 8 schematically illustrates a kit 800 for amending a foot, according to exemplary embodiments of the disclosed subject matter.

Kit 800 comprises one or more of elevation 102 and one or more of elevation 104.

Elevation 102 and elevation 104 are illustrated with different shapes to indicate an optional different adaptation to the metatarsal bones and the heel, respectively.

A dashed elevation 802 and a dashed elevation 804 as well as a dashed line 812 and a dashed line 814 represent optional additional instances of elevation 102 and elevation 104, respectively.

In some embodiments, kit 800 optionally comprises one or more fasteners such as band 112 and band 114, and in some embodiments, kit 800 comprises one or more of elastic members such as strap 106 and/or strap 108 (not shown).

It is emphasized that kit 800 may comprise only one elevation for applying foot warping as described above with respect to a single elevation.

In some embodiments, only a single elevation instead of two or more are used, such as at the front of the foot or the rear of the foot. In some embodiments, the single elevation comprises a multiplicity of elevating members such as an inflatable elevation with a plurality of compartments as described above.

It is noted that, at least generally, the effect of warping the foot increasing and/or decreasing the curvature of the arch by the elevations exists also when the person walks where the heel is laid down to subsequently lay the toes and raise the heel.

It is noted that the apparatus and methods described herein provide a therapy external to the foot, and furthermore, the person applies the therapy himself as he stands or walks or runs.

There is thus provided according to the present disclosure a method for amending a foot of a person, comprising disposing elevations under the foot, wherein an at least one first elevation is disposed under an at least one metatarsal bone of the foot and an at least one second elevation is disposed lengthwise diagonally under the heel of the foot, and applying a downwards force on the foot, thereby warping the foot over the elevations and amending the foot arch.

In some embodiments, applying a downwards force is due to the person's weight while: standing on the foot, walking, running, or any combination thereof.

In some embodiments, the elevations disposed under the foot allow the person to wear shoes and conduct daily routines.

In some embodiments, the at least one first elevation is disposed under at least one of the 5th metatarsal bone, the 4th metatarsal bone, the 3rd metatarsal bone, or any combination thereof, and the at least one second elevation is disposed under the heel or the side thereof obliquely from the at least one first elevation; and wherein amending the foot arch comprises increasing the curvature of the foot arch.

In some embodiments, a increasing the curvature of the foot arch comprises increasing the curvature of the arch or a flat foot.

In some embodiments, the at least one first elevation is disposed under at least one of the 1st metatarsal bone, the 2nd metatarsal bone, the 3rd metatarsal bone, or any combination thereof, and the at least one second elevation is disposed under the heel or the side thereof obliquely from the at least one first elevation; and wherein amending the foot arch comprises decreasing the curvature of the foot arch.

In some embodiments, decreasing the curvature of the foot arch reduces, at least partially, an at least one of disorder syndromes of the foot.

In some embodiments, the at least one of disorder syndromes of the foot comprises at least one of: inflammation and thickening of plantar fascia, compression of nerves adjacent to fascia in Tarsal Tunnel Syndrome, enthesopathy Tendo Achilles, or any combination thereof.

In some embodiments, the method further comprises fastening the elevations to the foot to maintain the positions thereof.

In some embodiments, the method further comprises applying an at least one elastic member on the foot to improve at least one of a posture of the foot or a stability of the foot.

In some embodiments, applying the at least one elastic member on the foot effects at least one of stimulating or activation of muscles of the foot, thereby strengthening the muscles.

In some embodiments, applying the at least one elastic member on the foot comprises at least one of: wrapping an elastic strap around the foot, wrapping an elastic strap over the foot, wearing an elastic sock, or wearing a part of an elastic sock, or any combination thereof.

In some embodiments, disposing elevations under the foot is carried out repetitively with different elevations for gradual amendment of the foot.

In some embodiments, at least one of the at least one first elevation or the at least one second elevation comprises a plurality of elevations.

There is further provided according to the present disclosure an apparatus for amending a foot of a person, comprising an at least one elevation for disposing under the foot, and an at least one fastener for securing the elevations in place under the foot.

In some embodiments, the at least one elevation comprises a plurality of elevations.

In some embodiments, the apparatus further comprises an at least one elastic member for applying on the foot or above the foot to improve at least one of a posture of the foot or a stability of the foot.

In some embodiments, the at least one elevation comprises an inflatable compartment.

There is yet further provided according to the present disclosure a kit for amending a foot of a person, comprising an at least one elevation for disposing under the foot, and an at least one fastener for securing the elevations in place under the foot.

In some embodiments, the at least one elevation comprise a plurality of elevations.

In some embodiments, the kit further comprises an at least one elastic member for applying on the foot or above the foot to improve at least one of a posture of the foot or a stability of the foot.

There is yet further provided according to the present disclosure a method for amending a foot of a person, comprising disposing an at least one elevation under the foot, disposed under an at least one metatarsal bone of the foot or disposed under the heel of the foot, and applying a downwards force on the foot, thereby warping the foot over the elevations and amending the foot arch.

in some embodiments, applying the downwards force is due to the person's weight while: standing on the foot, walking, running, or any combination thereof.

In some embodiments, the at least one elevation disposed under the foot allows the person to wear shoes and conduct daily routines.

In some embodiments, depending on the position the at least one elevation the amending the foot arch comprises either increasing the curvature of the foot arch or decreasing the curvature of the foot arch.

In some embodiments, decreasing the curvature of the foot arch reduces, at least partially, an at least one of disorder syndromes of the foot.

In some embodiments, the method further comprises fastening the at least one elevation to the foot to maintain the position thereof.

In some embodiments, the method further comprises comprising applying an at least one elastic member on the foot to improve at least one of a posture of the foot or a stability of the foot.

In some embodiments, applying the at least one elastic member on the foot effects at least one of stimulating or activation of muscles of the foot, thereby strengthening the muscles.

In some embodiments, disposing the at least one elevation under the foot is carried out repetitively with different elevations for gradual amendment of the foot.

The flowchart and block diagrams illustrate architecture, functionality or an operation of possible implementations of systems, methods and computer program products according to various embodiments of the present disclosed subject matter. In this regard, each block in the flowchart or block diagrams may represent a module, segment, or portion of program code, which comprises one or more executable instructions for implementing the specified logical function(s). It should also be noted that, in some alternative implementations, illustrated operations may occur in deferent order or as concurrent operations instead of sequential operations to achieve the same or equivalent effect.

The corresponding structures, materials, acts, and equivalents of all means or step plus function elements in the claims below are intended to include any structure, material, or act for performing the function in combination with other claimed elements as specifically claimed. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The terminology used herein is for the purpose of describing particular embodiments only and is not intended to be limiting of the disclosed subject matter. While certain embodiments of the disclosed subject matter have been illustrated and described, it will be clear that the disclosure is not limited to the embodiments described herein. Numerous modifications, changes, variations, substitutions and equivalents are not precluded.

1-32. (canceled)

32. A method for affixing an orthotic for a foot of a person, comprising:
wearing a sock (170) having elevations fastened thereto and comprising an at least one first elevation (102) disposed in a position corresponding to beneath an at least one metatarsal bone of the foot, and an at least one second elevation (104) disposed in a position corresponding to beneath the heel of the foot correspondingly lengthwise diagonally with respect to the length of the foot; and
securing the at least one first elevation under the at least one metatarsal bone and the at least one second elevation in place under the heel by stretching the sock on and over the foot along the length of the foot.

33. The method according to claim 32, wherein the sock is configured for warping the foot over the elevations due to a downwards force on the foot ensuing from the person's weight, thereby gradually effecting remedy of the foot arch.

34. The method according to claim 32, wherein the sock is configured for allowing the person to wear shoes and conduct daily routines while wearing the sock.

35. The method according to claim 32, wherein the at least one first elevation is secured under at least one of: the 5th metatarsal bone, the 4th metatarsal bone, the 3rd metatarsal bone, or any combination thereof,
and the at least one second elevation is secured under the heel or the side thereof obliquely from the at least one first elevation,
for gradually effecting increasing the curvature of the foot arch.

36. The method according to claim 32, wherein the at least one first elevation is secured under at least one of: the 1st metatarsal bone, the 2nd metatarsal bone, the 3rd metatarsal bone, or any combination thereof,
and the at least one second elevation is secured under the heel or the side thereof obliquely from the at least one first elevation,
for gradually effecting decreasing the curvature of the foot arch.

37. The method according to claim 32, wherein the sock comprises a stretchable elastic fabric.

38. The method according to claim 32, wherein the sock is configured for improving at least one of a posture of the foot or a stability of the foot.

39. The method for amending a foot of a person according to claim 32, wherein at least one of the elevations comprises a plurality of elevations.

40. An orthotic apparatus affixable on a foot of a person, comprising:
a sock (170) configured for stretching on and over the foot along the length of the foot;
elevations fastened to the sock and securable under the foot by the sock, comprising:
an at least one elevation (102) attached to the sock in a position corresponding to beneath an at least one metatarsal bone of the foot; and
an at least one second elevation (104) attached to the sock in a position corresponding to beneath the heel of the foot correspondingly lengthwise diagonally with respect to the length of the foot.

41. The apparatus according to claim 40, wherein the sock comprises a stretchable elastic fabric.

42. The apparatus according to claim 40, wherein the sock is configured for warping the foot over the elevations due to a downwards force on the foot ensuing from the person's weight, thereby gradually effecting remedy of the foot arch.

43. The apparatus according to claim 40, wherein the sock is configured for allowing the person to wear shoes and conduct daily routines while wearing the sock.

44. The apparatus according to claim 40, wherein the at least one first elevation is securable under at least one of: the 5th metatarsal bone, the 4th metatarsal bone, the 3rd metatarsal bone, or any combination thereof,
and the at least one second elevation is securable under the heel or the side thereof obliquely from the at least one first elevation,
for gradually effecting increasing the curvature of the foot arch.

45. The apparatus according to claim 40, wherein the at least one first elevation is securable under at least one of: the 1st metatarsal bone, the 2nd metatarsal bone, the 3rd metatarsal bone, or any combination thereof,
and the at least one second elevation is securable under the heel or the side thereof obliquely from the at least one first elevation,
for gradually effecting decreasing the curvature of the foot arch.

46. The apparatus according to claim 40, wherein the at least one of the elevations comprises a plurality of elevations.
